(19) United States
(12) Patent Application Publication
(10) Pub. No.: US 2024/0215846 A1
(43) Pub. Date: Jul. 4, 2024

(54) CALIBRATION SYSTEM FOR AN ESOPHAGUS CATHETER WITH A BALLOON PROBE FOR DETERMINING ESOPHAGEAL PRESSURE

(71) Applicant: Hamilton Medical AG, Bonaduz (CH)

(72) Inventor: David Schwenninger, Felsberg (CH)

(73) Assignee: Hamilton Medical AG, Bonaduz (CH)

(21) Appl. No.: 18/279,682

(22) PCT Filed: Feb. 18, 2022
(86) PCT No.: PCT/EP2022/054098

§ 371 Date: Jan. 31, 2024

(30) Foreign Application Priority Data
Mar. 2, 2021 (DE) 10 2021 104 993.8

(51) Int. Cl. A61B 5/03; A61B 5/00

(52) U.S. Cl. CPC A61B 5/037; A61B 5/6853; A61B 2560/0223

(57) ABSTRACT

The invention relates to a calibration system for automatically setting an intended operational filling of an esophageal catheter with balloon probe, which can be inserted into the esophagus, for determining an esophageal pressure, in particular for a ventilation device, comprising a device for filling the balloon probe with a measuring fluid after placing the balloon probe in the esophagus, a pressure sensor for detecting the esophageal pressure prevailing in the balloon probe, and a calibration controller which is designed such that it incrementally changes the amount of measuring fluid in the balloon probe, the calibration controller recording an esophageal pressure detected by the pressure sensor for each amount of measuring fluid in the balloon probe set incrementally as a measuring point in this way and assigning said esophageal pressure to the respective set amount of measuring fluid in the balloon probe. The calibration controller is designed such that, in order to approach the respective measuring points, it monotonically changes the amount of measuring fluid in at least two steps, starting from a start value until an end value is reached.

### BACKGROUND

The present invention relates to a calibration system for an esophageal catheter with balloon probe for determining an esophageal pressure.

Esophageal catheters with balloon probes (hereinafter also briefly referred to as balloon catheters) for determining an esophageal pressure are used in particular in mechanical ventilation in order to determine the transpulmonary pressure.

In today's common forms of mechanical ventilation, the patient is supplied with breathing gas at a positive pressure. Therefore, during ventilation, the airway pressure or alveolar pressure is greater than the pressure in the pleural space or gap surrounding the pulmonary alveoli, at least during the inspiration phase. During the expiration phase, there is no pressurization of the airway by the ventilation device, with the result that the lung tissue relaxes and the airway pressure or alveolar pressure decreases. Under certain circumstances, this kind of positive pressure ventilation can cause the pressure conditions in the airway or in the alveoli to become so unfavorable at the end of the expiration phase that parts of the alveoli collapse. The collapsed part of the lung volume then must first be unfolded again in the subsequent breathing cycle. The functional residual capacity of the lungs is severely impaired, so that oxygen saturation decreases, and the lung tissue also suffers permanent damage.

To prevent collapse of alveoli at the end of the expiration phase, a so-called positive end-expiratory pressure, commonly briefly referred to as PEEP, is usually set during positive pressure mechanical ventilation. With this measure, an improvement in oxygen saturation can be achieved in many cases.

When ventilating with PEEP, the ventilation device applies a predetermined positive pressure, the PEEP, to the airway permanently—i.e. both during the inspiration phase and during the expiration phase. The PEEP is therefore still present after the end of the expiration phase.

Ideally, the PEEP should be set sufficiently high so that during the expiration phase the alveolar pressure is not, or at least only to such an extent, below the pressure in the pleural gap that the alveolar tissue does not collapse under the effect of the pressure in the pleural gap. In other words, the PEEP is to prevent the transpulmonary pressure—which is the pressure difference between alveolar pressure and pressure in the pleural gap—from becoming lower than zero or from falling below a lower negative limit value at which parts of the alveoli begin to collapse.

On the other hand, a too high value of the PEEP may have a negative effect, especially during the inspiration phase. This is because the lung tissue can become overstretched at very high airway pressures during the inspiration phase. Numerous studies also indicate that a high value of the PEEP can impede the return flow of venous blood to the heart with corresponding negative effects on the cardiovascular system.

In itself, the PEEP should be matched to the respective prevailing transpulmonary pressure. However, the transpulmonary pressure in a ventilated patient is not amenable to simple determination. To get by, one therefore measures the pressure in a balloon probe placed in the esophagus of a ventilated patient by means of an esophageal catheter. With suitable positioning and configuration of the balloon, the pressure measured in the balloon can be used to approximately determine the pressure in the pleural gap.

WO 2014/037175 A1 describes the automated setting of a pressure specified by a ventilation device, in particular the positive end-expiratory pressure (PEEP) and the maximum airway pressure, on the basis of a pressure detected by a measuring device, which is regarded as an indicator of the transpulmonary pressure, i.e. the pressure difference between alveolar pressure and pressure in the pleural gap. When the transpulmonary pressure is determined, the pressure in a balloon catheter inserted into the esophagus is measured as a measurand of the pressure in the pleural gap.

In practice, the relationship between the pressure measured in a balloon catheter inserted into the esophagus and the pressure in the pleural gap may change during ventilation. The causes for this can be manifold and generally cannot be determined in detail.

Mojoli et al, Crit. Care (2016) 20:98, describe a procedure for calibrating a balloon catheter designed to measure esophageal pressure. The aim of the calibration is to achieve optimal filling of the balloon catheter with air, in which the balloon catheter is as sensitive as possible to changes in the pressure in the pleural gap acting on the esophagus and reflects the pressure in the pleural gap as well as possible. Calibration is performed in vivo with the balloon catheter inserted into the esophagus. The pressure measured by the balloon catheter at the end of the inspiration phase and the pressure measured by the balloon catheter at the end of the expiration phase are measured successively with different amounts of fluid (air) filled into the balloon catheter, and the difference between the two pressures is determined. The optimal balloon filling is then obtained as the maximum of the pressure difference in a measuring range in which both the pressure measured by the balloon catheter at the end of the inspiration phase and the pressure measured by the balloon catheter at the end of the expiration phase increase quasi-linearly with the amount of fluid filled into the balloon. For being able to achieve reproducible results in this in vivo calibration, each balloon filling in this study was adjusted according to the same procedure, as follows: starting with an empty balloon catheter, in pressure equilibrium with the environment, the balloon catheter was first overfilled to a value above the measuring range and then fluid was drained from the balloon until the desired balloon filling was achieved. Care must always be taken to allow sufficient time to compensate for fluid flows, pressures, and/or for the occurrence of relaxation processes in the catheter material or esophageal tissue before recording a new measuring point, thus avoiding unstable conditions. The measurement procedure described in Mojoli et al. is therefore comparatively complex and sensitive, so that the calibration of a balloon catheter requires a considerable amount of time of approximately 15 min or even longer. In practice, this leads to the fact that only an optimal filling for the balloon probe can be determined and preset before the start of a ventilation process. This optimal filling is then maintained during ventilation and is not changed any more.

Hotz et al, Respir. Care (2018) 63(2): 177-186, also recommend calibration of the amount of fluid filled into the balloon catheter to obtain maximum sensitivity of the measured pressure to pressure changes in the pleural gap and improve the accuracy of the measurement. The in vivo calibration proposed therein is similar to the calibration proposed in Mojoli et al, and in particular is similarly time consuming.

### SUMMARY

The present invention provides a calibration system which achieves an improvement in the accuracy of pressure measurement values delivered by a balloon catheter inserted into the esophagus. In particular, the calibration system according to the invention allows more accurate reproduction of the pressure prevailing in the pleural gap by the balloon catheter and more rapid response of the measurement values to changing environmental conditions during ongoing ventilation. This enables calibration of the balloon catheter during ventilation without the need to interrupt ventilation. This is true even when ventilation is performed using fully automatic ventilation modes, such as closed-loop ventilation, as in Adaptive Support Ventilation (ASV ventilation) developed by the applicant.

According to the invention, a calibration system is proposed which is designed for automatically setting an intended operational filling of an esophageal catheter with balloon probe, which can be inserted into the esophagus, for determining an esophageal pressure, in particular for a ventilation device. The calibration system comprises the following components:
a filling device for filling the balloon probe with a measuring fluid after placing the balloon probe in the esophagus,
a pressure sensor for detecting an esophageal pressure prevailing in the balloon probe,
a calibration controller which is designed such that it incrementally changes the amount of measuring fluid in the balloon probe, the calibration controller recording an esophageal pressure detected by the pressure sensor for each amount of measuring fluid in the balloon probe set incrementally as a measuring point in this way and assigning the esophageal pressure to the respective set amount of measuring fluid in the balloon probe.

The calibration controller is designed such that, for approaching the respective measuring points, it monotonically changes the amount of measuring fluid in at least two steps, starting from a start value until an end value is reached.

The measuring fluid is in particular air.

In particular, the calibration system may also comprise a device for withdrawing measuring fluid from the balloon probe (fluid draining device) after placing the balloon probe in the esophagus. Both the device for filling the balloon probe with a measuring fluid after placing the balloon probe in the esophagus and the device for withdrawing measuring fluid from the balloon probe after placing the balloon probe in the esophagus may comprise one or more valves arranged in a conveying line for measuring fluid that is in fluid communication with the balloon probe. Insofar as the measuring fluid in the balloon probe is under positive pressure, the fluid draining device may be implemented simply by controlling valves in a fluid line in fluid communication with the balloon probe.

The device for filling the balloon probe with a measuring fluid after placing the balloon probe in the esophagus may comprise a pumping device by means of which measuring fluid may be pumped into the balloon probe. In this case, the pumping device may also be configured to withdraw (pump out) measuring fluid from the balloon probe. The fluid draining device may comprise the pumping device.

In a further embodiment, the device for filling the balloon probe with a measuring fluid and/or the device for withdrawing measuring fluid from the balloon probe may comprise a flow sensor, in particular a mass flow sensor, which is designed to determine an amount of measuring fluid introduced into the balloon probe and/or an amount of measuring fluid withdrawn from the balloon probe. For example, by integrating a flow measured by the flow sensor over a period of time between a start time and an end time, the respective amount of measuring fluid introduced into the balloon probe and/or withdrawn from the balloon probe can be determined. If a flow sensor is used to determine the flow of measuring fluid based on a differential pressure, the flow sensor can also be used to detect the esophageal pressure prevailing in the balloon probe. A separate pressure sensor is possible as well.

The calibration controller can be implemented as a stand-alone component “in hardware”. Alternatively, the calibration controller may be realized as a computer program product, i.e. by a corresponding software program executed on a processor, in particular a microprocessor or microcontroller. In this case, the software can be kept on a suitable local storage medium or a storage medium that can be retrieved via a network. The software contains instructions coded as a computer program which, when the software is loaded into a RAM memory of the processor and translated into machine language, causes the processor to execute the procedures described herein in more detail. Mixed forms between realization in hardware and realization in software are, of course, conceivable as well. The microprocessor or microcomputer may be part of a control system of the calibration system.

The term “monotonically” is intended to express that the amount of measuring fluid in the balloon probe always changes in the same direction during a measuring cycle. This means that the amount of measuring fluid either continues to decrease or continues to increase during a measuring cycle between the start value and the end value for reaching each further measuring point. In this context, the approaching of measuring points between the start value and the end value defines a measuring cycle. Start value and end value of a measuring cycle can be defined by predetermined amounts of measuring fluid introduced into or drained from the balloon probe. Alternatively, it is also conceivable to define the start value and end value by predetermined values of the pressure detected in the balloon probe. The same applies to the individual measuring points that are approached between the start value and end value of a measuring cycle. It has been found that it is simpler and faster, at least when approaching successive measuring points between the start value and the end value (wherein start and end values possibly are not included), to always discharge a predetermined amount of measuring fluid from the balloon probe or to introduce it into the balloon probe.

The monotonic traversal of the measuring range between the start value and the end value within a measuring cycle, as proposed according to the invention, accelerates the calibration enormously. This is in particular due to the fact that successive measuring points can be approached directly one after the other within a measuring cycle, starting from the start value up to at least the end value. In particular, there is no need for intermediate steps to evacuate the balloon probe and/or to set precisely reproducible initial conditions for the individual measuring points during a calibration process. In addition, there is no longer any need to wait for pressure equalization or relaxation processes. Surprisingly, it has turned out that calibration is not subject to any—at least not unacceptably large—negative effects by omitting, for each measuring point, an identical procedure with the same initial conditions for each measuring point. In particular, hysteresis effects—if any—appear to have approximately the same effect on all measuring points within a measuring cycle and therefore do not interfere with the calibration.

Special embodiments of the calibration system proposed herein may have one or more of the optional features set forth below. Where features relate exclusively to alternative embodiments, this will be expressly noted below. It is therefore understood that the following features may be combined in any manner with the respective features set forth above or below, unless expressly excluded.

As already mentioned, the calibration system may comprise furthermore a fluid draining device which is designed to drain measuring fluid from the balloon probe. The calibration controller controls the fluid draining device for approaching the respective measuring points in such a way that the amount of measuring fluid in the balloon probe decreases monotonically in at least two steps, starting from the start value until the end value is reached. The control can be carried out, for example, by temporarily opening valves in a conveying line that is in fluid communication with the balloon probe. Active pumping by means of a corresponding pumping device is also conceivable.

The calibration controller can further be designed to control the arrangement for filling the balloon probe with a measuring fluid at least in a first measuring cycle for filling the balloon probe with an amount of measuring fluid in such a way that the amount of measuring fluid introduced into the balloon probe is greater than an upper limit of the measuring range assigned to the measuring cycle between the start value and the end value. In this case, the start value, which defines the beginning of the measuring cycle, is only approached after a first amount of measuring fluid has been drained from the balloon probe. The calibration controller can control the arrangement for filling the balloon probe such that the latter initially fills the balloon probe with a larger amount of measuring fluid than the amount of measuring fluid corresponding to the start value. The calibration controller can then control the fluid draining device in such a manner that the latter drains measuring fluid from the balloon probe until the amount of measuring fluid corresponding to the start value is present in the balloon probe.

These measures lead to certain overstretching of the balloon probe before the start of the upcoming measuring cycle. This ensures that the measuring cycle actually covers the entire range that can be used as a measuring range, in which the pressure measured by the balloon probe changes approximately linearly with the amount of measuring fluid in the balloon probe. In the proposed procedure with overstretching of the balloon probe before approaching the start value, the pressure measured in the balloon probe thus decreases approximately linearly with the amount of measuring fluid in the balloon probe during the measuring cycle, at least in a middle portion. This linear decrease determines the range between an upper filling amount of measuring fluid in the balloon probe and a lower filling amount of measuring fluid in the balloon probe, which can basically be used as a measuring range for the esophageal catheter.

Since the measurement is performed in vivo, i.e. with the balloon probe placed in the esophagus of a patient, the slope of the esophageal pressure/amount of measuring fluid curve in the linear part used as the measuring range is determined more by the distensibility of the esophagus and less by the distensibility of the balloon probe. In the linear part, the esophagus stretches less and less as the amount of measured fluid decreases, and its cross-section decreases as the amount of measured fluid decreases while the distensibility remains approximately the same.

In the range above the linear part of the measuring range—at least with a sufficiently large diameter of the balloon probe—the maximum distensibility of the esophagus is reached. The balloon probe can then hardly stretch any further with a further increase in the amount of fluid filled in. Therefore, the pressure in the esophageal balloon increases more steeply with increasing measuring fluid volume than in the linear part of the measuring range. As a rule, at least in a first measuring cycle, the start value for the amount of measuring fluid will be selected such that the start value for the measured esophageal pressure is in the range above the linear part of the measuring range.

In a further embodiment, the calibration controller can be designed to perform at least two measuring cycles in succession. In this case, the measuring range of the at least two successive measuring cycles can be different. In particular, a preceding measuring cycle can determine the measuring range for a subsequent measuring cycle.

For example, in the preceding measuring cycle, a first measuring range between a first start value and a first end value can be traversed in order to find approximately the linear range in which the distensibility of the esophagus remains approximately the same. Then, in the subsequent measuring cycle, a finer gradation of measuring points can be made between a second start value and a second end value, both of which are in the linear range.

The calibration controller may further be designed to set the distance between successive measuring points differently for the preceding measuring cycle and for the subsequent measuring cycle. In this context, the term “distance between measuring points” is intended to refer to the difference between the associated amounts of measuring fluid in the balloon probe.

For example, in a preceding measuring cycle, a relatively wide distance between successive measuring points may be set to identify the range in which the esophageal pressure detected in the esophageal catheter changes approximately linearly with the amount of measuring fluid in the balloon probe. In a subsequent measuring cycle, this linear range between the associated maximum amount of measuring fluid and minimum amount of measuring fluid can then be traversed with a smaller step width in order to find the optimal filling amount of measuring fluid for the esophageal catheter.

Furthermore, the calibration controller may be designed to adaptively determine the step width or increments between successive measuring points within the measuring range in a measuring cycle. Such adaptive increment determination can be performed, for example, using a gradient method such as Newton's algorithm.

In the case of several successive measuring cycles, it may be advantageous to carry out the certain “overstretching” of the balloon probe at the beginning, i.e. to perform the initial introduction of a larger amount of measuring fluid than corresponds to the start value of the respective measuring cycle, before each measuring cycle. In that case, the calibration controller may be designed such that no complete emptying of the measuring fluid from the balloon probe takes place between a preceding measuring cycle and a subsequent measuring cycle. In such embodiments, in particular, there is no complete evacuation of the cavity or volume enclosed by the balloon probe, not even between successive measuring cycles. Rather, one makes use of the fact that the exact amount of measuring fluid present in the balloon probe before the start of the respective measuring cycle is not important if, starting from an arbitrary start value at the beginning, a larger amount of measuring fluid is introduced into the balloon probe than is required for the start value, because the overstretching thus effected depends much more on the amount of measuring fluid introduced into the balloon probe than on the initial state from which the filling started. In addition, the exact degree of overstretching of the balloon probe is often not important at all, because when the measuring fluid is withdrawn from the balloon probe, which is necessary to reach the start value for the subsequent measuring cycle, the desired start value for the respective measuring cycle can be recognized from the detected esophageal pressure.

However, for improving accuracy, it may also be envisaged to set the balloon probe to a predetermined “zero state” before the start of a measuring cycle, or at least before the start of a first measuring cycle in the case of several successive measuring cycles, for example by pumping or otherwise draining measuring fluid from the balloon probe until a predetermined negative pressure (e.g. −20 hPa relative to ambient pressure) is reached. In this way, it can be ensured that the calibration procedure starts from a predetermined initial state of the balloon probe, in which, for example, it is certain that the balloon probe has a fully collapsed configuration. Starting from this zero state, measuring fluid can then be introduced or pumped into the balloon probe until overfilling of the balloon probe to a predetermined pressure that is above the start value for the first measuring cycle has occurred. From this state, the calibration procedure can then be performed in the manner described above by successively approaching a plurality of measuring points monotonically between a start value and an end value.

In further embodiments, the calibration controller may be designed such that for each measuring point, i.e. for each set amount of measuring fluid in the balloon probe between the start value and the end value of a measuring cycle (if desired, including the start value and the end value), it ascertains a measurement value for the esophageal pressure at the end of an inspiration phase and a measurement value for the esophageal pressure at the end of an expiration phase and then determines the difference between the esophageal pressure at the end of the inspiration phase and the esophageal pressure at the end of the expiration phase.

In this regard, the calibration controller may be designed to determine a maximum value for the difference between the esophageal pressure at the end of the inspiration phase and the esophageal pressure at the end of the expiration phase (maximum pressure) for a range between the start value for the amount of measuring fluid in the balloon probe and the end value for the amount of measuring fluid in the balloon probe (if desired, including the start value and the end value). The amount of measuring fluid in the balloon probe corresponding to the maximum pressure then corresponds to the optimal filling for the balloon probe, i.e. the amount of measuring fluid in the balloon probe with which ventilation should be performed.

Furthermore, the calibration controller may be designed such that it ascertains the measurement value for the esophageal pressure at the end of an inspiration phase and the measurement value for the esophageal pressure at the end of an expiration phase for a respective measuring point between the start value and the end value during ongoing ventilation.

An ongoing ventilation cycle or breathing cycle therefore does not need to be interrupted to ascertain or determine a measurement value for the esophageal pressure. In particular, to ascertain these measurement values, it is not necessary to set a dead time during which the airways are closed so that the flow of ventilation gas or breathing gas comes to a standstill. Thus, ventilation of a patient can continue unchanged while calibration or recalibration of the esophageal catheter is performed. This is a great advantage, not only because any interruption or disturbance of ventilation is to be avoided from the patient's point of view, but also because calibration of the esophageal catheter can be performed in this way under conditions as close to life as possible.

Furthermore, the calibration controller can be designed such that it compares the differences determined for the respective measuring points between the esophageal pressure at the end of the inspiration phase and the esophageal pressure at the end of the expiration phase, and then, if the pressure difference determined for a respective measuring point lies within a predetermined fluctuation range with respect to a maximum value of the pressure difference across a plurality of successive measuring points, it determines the amount of measuring fluid corresponding to optimal filling of the balloon probe as an amount of measuring fluid which has a predetermined distance from the smallest amount of measuring fluid in the balloon probe (lower edge) and/or from the largest amount of measuring fluid in the balloon probe (upper edge) at which the detected pressure difference still lies within the fluctuation range.

For example, it may be specified that the amount of measuring fluid corresponding to the optimal filling of the balloon probe is closer to the lower edge of such a range with approximately equal pressure differences near the maximum value than to the upper edge. For example, the amount of measuring fluid in the balloon probe corresponding to the optimal filling of the balloon probe (“optimal filling”) may be greater than the amount of measuring fluid corresponding to the lower edge of the range of approximately equal pressure difference by ⅓ of the distance between the amount of measuring fluid in the balloon probe corresponding to the upper edge of the range of approximately equal pressure difference and the amount of measuring fluid in the balloon probe corresponding to the lower edge of the range of approximately equal pressure difference.

In particular, the calibration controller can be designed such that it ascertains, for each measuring point between the start value and the end value (possibly including start value and end value), a plurality of measurement values, in particular a plurality of pairs of measurement values for the esophageal pressure at the end of an inspiration phase and for the esophageal pressure at the end of an expiration phase. This is usually done in successive ventilation cycles. The calibration controller can then determine an average and a statistical dispersion or scatter for the measurement value or a variable derived therefrom, in particular the difference between the esophageal pressure at the end of the inspiration phase and the esophageal pressure at the end of the expiration phase (pressure difference), on the basis of the plurality of measurement values for each measuring point, and can determine the number of measurements per measuring point such that the average obtained can be regarded as statistically significant.

As a general rule, the greater the statistical variation of the measurement values obtained, the more breathing cycles are used as a basis for determining the measurement value for the pressure difference assigned to a respective measuring point. Cardiogenic oscillations or movements, for example, can play a role here. Such effects can be better distinguished by increasing the number of breathing cycles per measurement. In particular, an arithmetic mean can be taken as the average. However, other types of averaging are also conceivable, for example a geometric average.

The statistical dispersion can be represented in particular by a Gaussian standard deviation. It would then be conceivable to define a 2-sigma level as statistically significant, i.e. to rate a Gaussian standard deviation of 95% or better as significant. Another variant could be to proceed adaptively for the determination of the dispersion, for example by using the change of the average value after each new measurement value as a measure of significance for a respective measuring point. The smaller the change of the average value after a respective measurement value, the higher the significance. Measurements can be repeated for a respective measuring point until the change of the average value after the last measurement has become smaller than a predetermined threshold value. Thereafter, it is possible to proceed to the next measuring point, such as by withdrawing a predetermined amount of measuring fluid from the balloon probe.

In further embodiments, the calibration controller can be designed to monitor, for each measuring point between the start value and the end value, whether the respective measurement (in the case of multiple measurements of a pressure difference per measuring point at each such measurement) is affected by external circumstances, and to discard the respective measurement if such external circumstances are detected. External circumstances can be, for example, swallowing efforts of a patient. It may be provided that not the entire calibration is discarded or aborted, but only the measurement value affected by external circumstances is discarded. In particular, a new measurement value for the respective measuring point can be ascertained immediately thereafter. After that, the calibration procedure can continue normally. Artificial measurement values are thus eliminated “in real time” without the calibration procedure having to be aborted or interrupted for longer periods of time. Restarting the entire calibration procedure after detection of external circumstances would take significantly longer and could, for example in the case of swallowing efforts by the patient during one or more measurements, also lead to renewed swallowing efforts being triggered in the patient. This is an enormous progress compared to known approaches, because with known approaches either the decision of an operator is required, which makes any automation impossible, or automated calibrations have to be carried out without considering external circumstances. A calibration is completely discarded only afterwards, if an analysis of the data reveals that at least one or some of the measurement values, which flowed into the calibration in question, are possibly affected by external influences such as swallowing efforts or the like. It is therefore not possible to say with certainty and already before the start of a calibration whether this calibration is valid or may have to be discarded.

Unusual external circumstances such as swallowing efforts by the patient can be detected, for example, by monitoring the time course of the esophageal pressure, in particular if changes in the pressure signal occur at the end of the machine-specified inspiration phase and/or at the end of the machine-specified expiration phase.

The calibration controller can be designed such that it interrupts the calibration when such disturbances occur and only resumes calibration when it can be determined from the detected esophageal pressure and/or pressure in the airway that no further disturbances occur.

Furthermore, the calibration controller may be designed to calculate a quality index based on the data acquired during the calibration procedure. The quality index may represent a weighted summary of the influences of various factors. In such embodiments, the quality index may be used to decide whether or not to allow changes in ventilation parameters based on esophageal pressure data. Criteria relevant to the quality index may include the statistical scatter of the measurement data or an unusual course of the esophageal pressure/amount of measured fluid curve. As a measure for the quality index, the scatter of the measurement values, for example expressed as Gaussian standard deviation, can be used. In addition, certain rules can be determined that are included in the quality index. Examples of such rules can be: if swallowing by the patient is detected during a calibration procedure, the quality index is reduced. If the maximum of the difference between the detected esophageal pressure at the end of inspiration and the detected esophageal pressure at the end of expiration at a measuring point is outside, especially above, the linear range of the curve for the relationship between esophageal pressure and amount of measuring fluid in the balloon probe, the quality index is reduced. If the baseline pressure, i.e. the esophageal pressure at the end of the expiration phase does not remain the same or change steadily over the course of successive measurements but changes in jumps, the quality index is reduced. If an esophageal pressure detected at a higher value of the fluid amount in the balloon probe is smaller than the value at the lower fluid amount in the balloon probe, the quality index is reduced.

Furthermore, the calibration controller may be designed such that the esophageal pressure must not exceed a predetermined maximum pressure. For example, it may be provided that the esophageal pressure at maximum must not be more than twice the maximum airway pressure during ventilation. When this pressure is reached, no further fluid is introduced into the balloon probe or fluid is drained from the balloon probe. This is to avoid excessive overstretching of the esophageal tissue. Also, excessive overstretching of the balloon probe, which could result in damage thereof, can be avoided in this manner.

Finally, it can be provided that the calibration controller controls a draining device already mentioned above, through which measuring fluid can be drained from the balloon probe, when approaching the respective measuring points such that the amount of measuring fluid in the balloon probe is incrementally reduced further and further, starting from the start value until the end value is reached when a predetermined minimum end-expiratory esophageal pressure, for example −5 hPa, is reached or is fallen short of.

The present invention further relates to a method for automated calibration of an intended operational filling of an esophageal catheter with balloon probe, which can be inserted into the esophagus, for determining an esophageal pressure, in particular for a ventilation device.

This method comprises the following steps:
filling the balloon probe with a measuring fluid after placing the balloon probe in the esophagus, and
detecting an esophageal pressure prevailing in the balloon probe, and
incrementally changing an amount of measuring fluid in the balloon probe, wherein for each amount of measuring fluid in the balloon probe set incrementally as a measuring point in this way, the esophageal pressure is detected and is assigned to the respective set amount of measuring fluid in the balloon probe.

In the method according to the invention, the amount of measuring fluid is changed monotonically in at least two steps, starting from a start value until an end value is reached, in order to approach the respective measuring points.

In further embodiments, the method according to the invention may comprise at least one, in particular several, of the further method steps implicitly mentioned above with reference to a formation of a calibration system. To avoid repetitions, express reference is made to the detailed explanation of these method steps with respect to functional features of the calibration system, in particular of the calibration controller.

Furthermore, the present invention relates to a computer program product which contains program instructions, during the execution of which on a data processing system, in particular on a microprocessor or a microcontroller for controlling an esophageal catheter with balloon probe, a calibration method according to the invention is carried out or a calibration system according to the invention is implemented.

Further embodiments of the present invention will be explained in more detail in the following with reference to the figures.

### BRIEF DESCRIPTION OF THE DRAWINGS

FIG. 1 shows in highly schematized form the essential elements of a ventilation device along with the intubated trachea and thorax of a ventilated patient;

FIG. 2 shows the time course of the airway inlet pressure Paw (top), esophageal pressure Peso (middle) and the difference Paw−Peso from both pressures during several consecutive breathing cycles during mechanical ventilation including occlusion maneuvers;

FIG. 3 shows, by way of a flowchart, the sequence of a calibration process of an esophageal catheter in vivo according to an embodiment of the present invention;

FIG. 4 schematically shows the amount of measuring fluid set in the balloon probe of the esophageal catheter during a calibration process according to an embodiment of the present invention, as well as the detected pressure in the balloon probe of the esophageal catheter; and

FIG. 5 schematically shows in partial images a) and b), for each set amount of measuring fluid in the balloon probe during a measuring cycle, the respective detected pressure in the balloon probe at the end of the inspiration phase, the respective detected pressure in the balloon probe at the end of the expiration phase (partial image a)), and the respective resulting differential pressure between the pressure in the balloon probe at the end of the inspiration phase and the pressure in the balloon probe at the end of the expiration phase (partial image b)).

### DETAILED DESCRIPTION

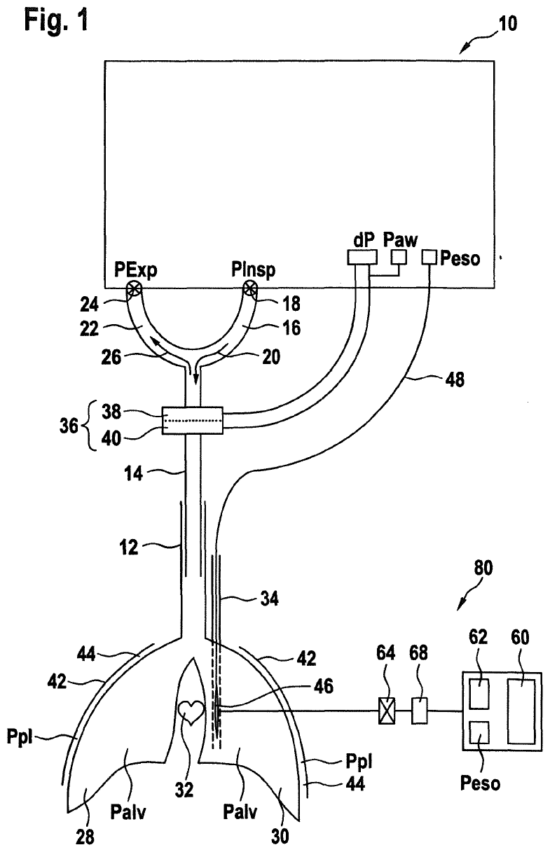

FIG. 1 illustrates the essential elements of a ventilation device 10 in a highly schematized manner and in the form of a block diagram. The ventilation device 10 is shown in FIG. 1 in a state with intubated trachea 12 of a ventilated patient. In addition to the trachea 12, the lung lobes 28, 30, the heart 32, the esophagus 34 and the thoracic wall 42 of the patient are shown very schematically in FIG. 1. The tube 14 of the ventilation device 10 is inserted, generally through the patient's mouth opening which is not shown, a short distance into the trachea 12 to supply breathing gas to the airway. Exhaled air is also discharged through tube 14, which branches at its upstream end into a first end 16 and a second end 22. The first end 16 is connected via an airway inlet valve 18 to an airway inlet port of the ventilation device 10 for applying an inspiration pressure PInsp. In the open position of the airway inlet valve 18, the inspiration pressure PInsp is applied to the airway. The second end 22 is connected via an airway outlet valve 24 to an airway outlet port of the ventilation device 10 for application of an expiration pressure PExp. In the open position of the airway outlet valve 24, the expiration pressure PExp is applied to the airway.

Both the inspiration pressure PInsp and the expiration pressure PExp are generated by the ventilation device 10 according to predetermined time patterns, such that inspiratory breathing gas flows toward the patient's lungs 28, 30 during an inspiration phase, as indicated by arrow 20 in FIG. 1, and expiratory breathing gas flows back from the patient's lungs 28, 30 during an expiration phase, as indicated by arrow 26. Normally, the airway inlet valve 18 remains open during the inspiration phase, and the inspiration pressure PInsp—which is usually greater than the expiration pressure PExp—is applied to the airway inlet. During the expiration phase, the airway inlet valve 18 is closed and the airway outlet valve 24 is open. Then, the expiration pressure PExp is applied to the airway inlet.

In the context of the present invention, any forms of known ventilation modes can be used, for example, pressure-controlled ventilation modes, volume-controlled ventilation modes, or ventilation modes in which pressure-controlled and volume-controlled aspects are combined. In addition to purely machine-controlled forms of ventilation, in which the time course of the inspiration pressure PInsp and possibly also of the expiration pressure PExp are determined by the ventilation device 10, it is also conceivable to have forms of ventilation in which the patient's spontaneous breathing efforts can either support the machine ventilation or the machine ventilation serves to support the patient's spontaneous breathing efforts. In such forms of ventilation, the time course of inspiration pressure PInsp or expiration pressure PExp and frequently also the position of inlet valve 18 or outlet valve 24 are not determined solely by the ventilation device 10, but are influenced by the patient's spontaneous breathing efforts.

The calibration of an esophageal catheter with balloon probe, as proposed according to the invention, which can be introduced into the esophagus, for detecting an esophageal pressure by means of which the transpulmonary pressure can be inferred, is particularly tailored to forms of ventilation in which ventilation is carried out by means of fully automatic ventilation modes, for example, ventilation by means of closed control loops, such as those used in the Adaptive Support Ventilation (ASV ventilation) developed by the applicant. Such forms of ventilation are characterized by the fact that only minimal manual intervention by the operator is required and that the ventilation device automatically sets or adjusts important ventilation parameters such as the positive end-expiratory pressure PEEP or the maximum airway pressure Paw_max within predefined value ranges using suitable closed control loops.

The breathing gas may contain ambient air, but will typically contain a predetermined proportion of pure oxygen, hereafter referred to as FiO2, which is above the oxygen content of ambient air. The breathing gas will also typically be humidified.

The flow of the breathing gas at the airway entrance or inlet is determined using an airway inlet flow sensor 36. The airway inlet flow sensor 36 is based on detecting a pressure difference dP between an input volume 38 and an output volume 40 in communication with the input volume 38, and provides a determination of the breathing gas mass flow at the airway inlet. At the same time, the value of the airway inlet pressure Paw can be derived quite easily from the pressure signal in the output volume 40.

The pressure prevailing in the alveoli of the lungs 28, 30 is indicated by Palv in FIG. 1. This pressure depends on the airway inlet pressure Paw as well as the flow of the breathing gas V into or out of the lungs and the airway resistance R. In the case of pressure equalization between the airway inlet and the alveoli, the alveolar pressure Palv is equal to the airway inlet pressure. Such a pressure equalization has the consequence that the breathing gas flow V comes to a standstill. For example, a brief occlusion maneuver of the airway, i.e. airway inlet valve 18 and airway outlet valve 24 remain closed at the same time, can lead to pressure equalization. In this case, the occlusion maneuver must last just long enough for the gas flow V in the airway to stop. This is usually between 1 and 5 s. In this state, the alveolar pressure Palv can then be determined by determination of the airway inlet pressure Paw.

Both in physiological breathing and in mechanical ventilation, the flow of breathing gas is determined by a pressure difference between the alveolar pressure Palv and the airway inlet pressure Paw.

In the case of purely physiological breathing, a negative pressure difference, i.e. a negative pressure, between the alveolar pressure Palv and the airway inlet pressure Paw is generated for inhalation by expansion of the thorax (indicated at 42 in FIG. 1) and associated lowering of the pressure Ppl in the pleural space or gap 44 formed between the thorax 42 and the lungs 28, 30. Exhalation takes place passively by relaxation of the thorax and elastic resetting of the lung tissue. For this reason, during physiological breathing, the pressure in the pleural gap Ppl is always lower than the alveolar pressure Palv. The transpulmonary pressure Ptp, defined as the difference between the alveolar pressure Palv and the pressure in the pleural gap Ppl, is thus generally positive and becomes zero in the case of complete pressure equalization.

During mechanical ventilation, the breathing gas is pumped into the lungs at a positive pressure. For this reason, in mechanical ventilation, the airway inlet pressure Paw=PInsp is greater than the alveolar pressure Palv and the latter in turn is greater than the pressure in the pleural gap Ppl during the inspiration phase. It follows from these pressure relationships that the transpulmonary pressure Ppl in mechanical ventilation is positive during inspiration. During expiration, the airway inlet has an airway pressure Pexp applied thereto that is lower than the alveolar pressure Palv, so that breathing gas flows out of the alveoli. In the case of a very low airway pressure PExp, it may happen at the end of expiration, when very little gas is left in the lungs, that the pressure in the pleural gap Ppl exceeds the alveolar pressure Palv to such a high extent that part of the alveoli of the lungs collapse. The transpulmonary pressure Ptp is then negative.

Collapsing of the alveoli can be prevented by applying an additional positive pressure to the airway inlet also during the expiration phase. A positive airway pressure is then permanently applied to the airway inlet, i.e. during the inspiration phase and also during the expiration phase. This positive airway pressure is referred to as positive end-expiratory pressure or PEEP.

Consequently, the transpulmonary pressure Ptp is a suitable parameter for setting the PEEP. However, the transpulmonary pressure Ptp is not amenable to direct detection and cannot be determined from the pressures regularly detected during mechanical ventilation, as described above, either.

In FIG. 1, an additional balloon probe 46 for measuring the pressure in the esophagus 34, referred to as esophageal pressure Peso, is indicated in schematic manner. The balloon probe 46 is in the form of a balloon and is attached to a catheter 48 inserted into the esophagus 34. The balloon probe 46 abuts internally against the wall of the esophagus 34 and provides the pressure applied to the esophagus 34 at the location of the balloon probe 46. This pressure is a good approximation of the pressure Ppl in the pleural gap when the patient is positioned appropriately. The described balloon probe 46 for detecting the esophageal pressure Peso and the handling of this probe is described, for example, in Benditt J., Resp. Care, 2005, 50: pp. 68-77.

If one wishes to determine the transpulmonary pressure Ptp, one needs information about the alveolar pressure Palv in addition to the pressure in the pleural gap Ppl. A rather elegant possibility for determining the alveolar pressure at a given time t is to detect the breathing gas flow V(t), which can be done with the aid of the flow sensor 36. The alveolar pressure at the time t can then be inferred according to the relationship: Palv(t)=Paw (t)−R*V(t), where R denotes the airway resistance. For one and the same patient, the airway resistance is a variable that essentially does not change or changes only comparatively slowly and can be determined in accordance with methods known in the prior art. For example, reference should be made to Lotti I. A. et al, Intensive Care Med., 1995, 21: 406-413. Since the transpulmonary pressure at the end of the expiration phase Ptp_ee is of primary importance for determining a suitable PEEP, in connection with an automatic setting of the PEEP, a determination of the alveolar pressure Palv will be performed preferably at the end of the expiration phase, according to the formula:

Ptp_ee=Palv_ee−Peso_ee=Paw_ee−R*V_ee−Peso_ee.

The PEEP should then be set or adjusted such that Ptp_ee always remains positive, or at least never drops significantly below zero.

Unfortunately, the described method of determining the alveolar pressure Palv, which is quite easy to implement in an automated ventilation device 10, allows only a comparatively rough estimate of the appropriate PEEP. This is mainly due to the airway resistance R, which can only be estimated quite imprecisely and which, moreover, will indeed generally be subject to a certain trend over the course of a therapy.

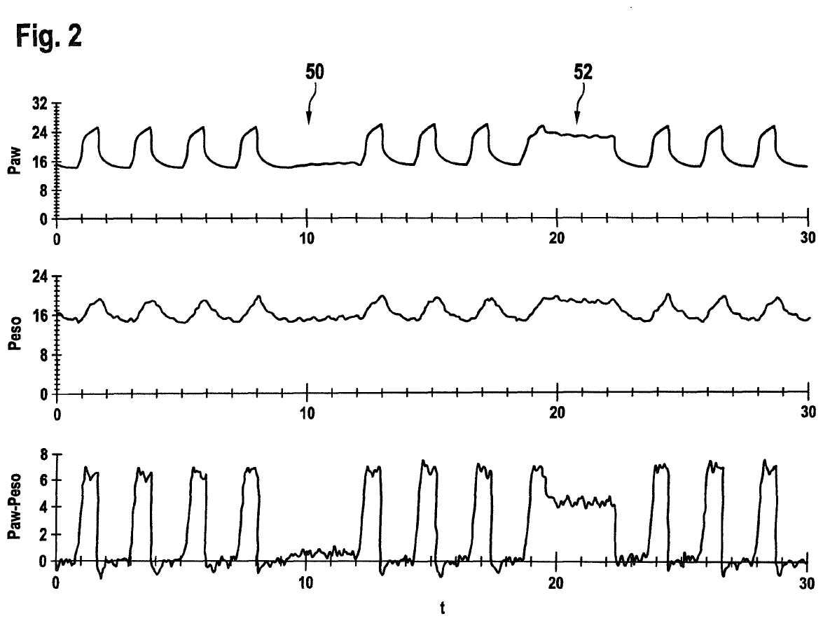

An alternative method for determining the alveolar pressure Palv is based on a brief occlusion maneuver in which both the airway inlet valve 18 and the airway outlet valve 24 remain closed at the same time. In this occlusion state, equalization of the pressures prevailing in the airway occurs. When such an occlusion maneuver is performed at the end of an expiration phase, the pressure established in the airway after a sufficiently long occlusion is, to a good approximation, equal to the alveolar pressure Palv at the end of the expiration phase. This pressure can then be detected quite easily using the pressure probe located at the airway entrance or inlet for measuring the airway pressure Paw. FIG. 2 shows this situation at the location designated 50.

FIG. 2 shows, one above the other, the time course of the airway pressure Paw (top), the esophageal pressure Peso measured by means of the balloon probe 46 (middle), and the difference Paw−Peso from both pressures during several successive breathing cycles, between which occlusion maneuvers were performed as well. One can clearly see the individual breathing cycles, each with an inspiration phase (high increasing airway pressure Paw) and an expiration phase (decreasing airway pressure Paw). The esophageal pressure Peso follows the airway pressure Paw, however in an attenuated form. The differential pressure Paw−Peso shown in the lower curve would correspond quite well to the transpulmonary pressure Ptp—given a sufficiently slow flow of the breathing gas so that pressure equalization always takes place. However, this condition is not the case in practice because of the highly variable flow of breathing gas, except at the locations designated 50 and 52, where a brief occlusion was performed at the end of an expiration phase (50, between about 8 s and 12 s) and a brief occlusion was performed at the end of an inspiration phase (52, between about 18.5 s and 24.5 s), respectively. In both cases, the occlusion lasted about 4 s. In the example chosen, this corresponds approximately to the duration of one breathing cycle. In general, occlusion should last long enough for pressure equalization to occur in the airway, thereby stopping the gas flow in the airway.

At the end of the location marked 50 in the time profile (approximately between 11 s and 12 s, for example, in the last approximately 200 ms of the occlusion), the pressure Paw−Peso shown in the third line in FIG. 2 corresponds in fairly good approximation to the transpulmonary pressure at the end of the expiration phase Ptp_ee. For determining Ptp_ee, one could, for example, average Paw−Peso over the period mentioned. At the end of the location marked 52 in the time profile (approximately between 21.5 s and 22.5 s, for example in the last approx. 200 ms of the occlusion), the pressure Paw−Peso shown in the third line corresponds in a fairly good approximation to the transpulmonary pressure at the end of the inspiration phase Ptp_ei. For determining Ptp_ei, one could, for example, average Paw−Peso over the mentioned period.

The determination of the transpulmonary pressure Ptp using the occlusion maneuver described is more accurate than the method described above using the airway resistance R. However, it requires performing an occlusion maneuver at the end of an expiration phase or at the end of an inspiration phase. Therefore, by its very nature, this method interferes with the breathing cycle, all the more significantly as the duration of occlusion is in comparison to the duration of the breathing cycle. For this reason, it is advisable to proceed in such a way that one checks quite frequently, for example after each breath or every n breaths (n>1), by means of the airway resistance method, whether a set value of the PEEP and/or a set value of the maximum airway pressure is still within the specifications or whether a resulting value of the transpulmonary pressure Ptp_ee is still within certain specifications for a normalized transpulmonary pressure Ptp_ee_ideal. If this examination reveals that this is not the case and that therefore a new (higher or lower) value for the PEEP and/or the maximum airway pressure should be set, an occlusion maneuver is performed in the following breathing cycle at the end of the expiration phase and the new value for the PEEP is determined on the basis of this occlusion as described above. Alternatively, the occlusion maneuver could be repeated every n breathing cycles as described, with n>1, for example, n=10, 50, or 100.

A prerequisite for the determination of the transpulmonary pressure Ptp using an esophageal catheter 48 with balloon probe 46, as described above, is that there is a fixed relationship between the transpulmonary pressure Ptp and the esophageal pressure Peso detected in the balloon probe 46. Ideally, the pressure Peso detected in the balloon probe 46 should correspond approximately to the pressure in the pleural gap Ppl, so that the transpulmonary pressure Ptp then results from the difference between airway pressure Paw and esophageal pressure Peso. In practice, however, it regularly happens that the relationship between the pressure Peso measured in a catheter 48 with balloon probe 46 inserted into the esophagus 34 and the pressure Ppl in the pleural gap changes during ventilation. Such changes usually cannot be clearly attributed to specific causes or events and often occur insidiously.

For this reason, calibration of such an esophageal catheter 48 would be desirable. However, ideas proposed in the prior art for calibrating esophageal catheters in vivo have proven to be extremely sensitive and time consuming. Therefore, these proposals are not particularly suitable for use during patient ventilation, especially for ongoing monitoring of esophageal catheters for proper calibration during ventilation.

The present invention provides a calibration system 80 that improves the accuracy of pressure measurement values provided by a catheter 48 inserted into the esophagus 34. In particular, the calibration system 80 according to the invention allows more accurate reproduction of the pressure Ppl prevailing in the pleural gap by way of the esophageal catheter 48 and more rapid response of the measurement values to changing environmental conditions during ongoing ventilation. This allows calibration of the esophageal catheter 48 during ventilation without having to interrupt ventilation to do so. This is true even when ventilation is performed using fully automatic ventilation modes, for example, closed-loop ventilation, such as the Adaptive Support Ventilation (ASV ventilation) developed by the applicant.

The calibration system 80 according to the invention includes a calibration controller 60. The calibration controller 60 is designed to incrementally change the amount of measuring fluid in the balloon probe 46, with the calibration controller 60 recording an esophageal pressure Peso for each amount of measuring fluid in the balloon probe 46 set incrementally as a measuring point in this way, and assigning said esophageal pressure to the respective set amount of measuring fluid in the balloon probe 46. For this purpose, the pressure signal Peso detected by the balloon probe 46 is also transmitted to the calibration controller 60.

The calibration system 80 comprises a pumping arrangement 62 for introducing measuring fluid into the balloon probe 46 and withdrawing measuring fluid from the balloon probe 46 after placing the balloon probe 46 in the esophagus 34. The pumping arrangement 62 includes a valve 64 disposed in a measuring fluid conveying line 66 in fluid communication with the balloon probe 46. In so far as the measuring fluid in the balloon probe 46 is at a positive pressure, the measuring fluid can be withdrawn from the balloon probe 46 simply by actuating the valve 64 without the aid of the actual pump.

The calibration system 80 further comprises a flow sensor 68, in particular a mass flow sensor, which is configured to determine an amount of measuring fluid introduced into the balloon probe 46 and/or an amount of measuring fluid withdrawn from the balloon probe 46. For example, by integrating a flow measured by the flow sensor 68 over a period of time between a start time and an end time, the respective amount of measuring fluid introduced into or withdrawn from the balloon probe 46 can be determined. For example, the flow sensor 68 may be configured to determine the mass flow of measuring fluid based on a differential pressure. In this case, the flow sensor 68 may also be used to detect the esophageal pressure Peso prevailing in the balloon probe 46.

The calibration controller 60 may be implemented as a stand-alone component “in hardware”. Alternatively, the calibration controller may also be realized as a computer program product, i.e. by a corresponding software program executed on a processor, in particular a microprocessor or microcontroller. In this case, the software can be kept on a suitable local storage medium or a storage medium that can be retrieved via a network. The software contains instructions coded as a computer program which, when the software is loaded into a RAM memory of the processor and translated into machine language, causes the processor to execute the procedures described herein in more detail. Mixed forms between realization in hardware and realization in software are, of course, conceivable as well. The microprocessor or microcomputer may associated with the control of the calibration system 80, and in particular may be part of the control of the calibration system 80.

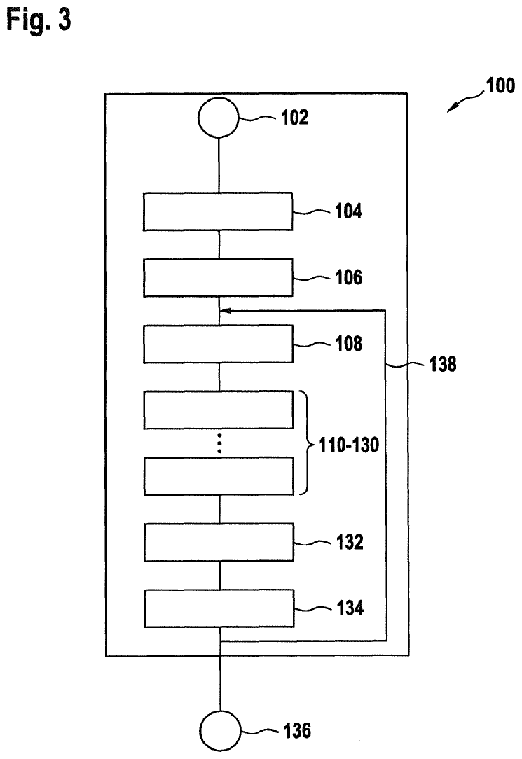

FIG. 3 illustrates, by means of a flowchart, the sequence of a calibration process of an esophageal catheter 48 in vivo according to an embodiment of the present invention.

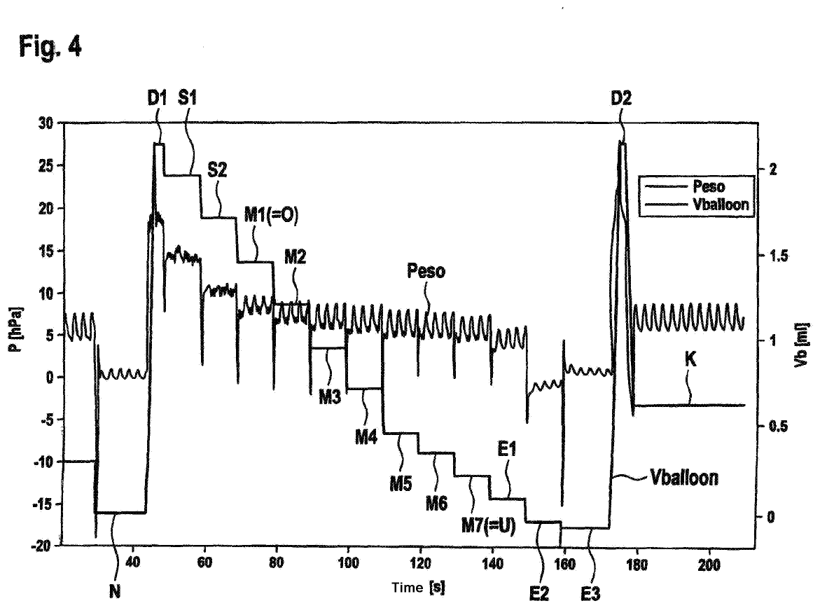

FIG. 4 schematically shows the time course of the amount of measuring fluid Vballoon set in the balloon probe 46 of the esophageal catheter 48 during a calibration process according to an embodiment of the present invention, as well as the detected pressure Peso in the balloon probe 46 of the esophageal catheter 48.

The calibration controller 60 is designed such that, for approaching the respective measuring points S1, S2, M1, M2, . . . , M7, E1, E2, E3, it changes the amount of measuring fluid Vballoon in the balloon probe 46 monotonically in at least two steps, starting from a start value S2 until an end value E3 is reached.

The measuring fluid is in particular air.

The calibration procedure 100 shown in FIG. 3 begins in step 102. First, in step 104, measuring fluid is pumped out of the balloon probe 46 until a predetermined initial pressure prevails in the balloon probe 46. The state reached in step 104 is designated “N” in FIG. 4.

Thereafter, in step 106, measuring fluid is again pumped into balloon probe 46 until a predetermined positive pressure exists in balloon probe 46 that is above the expected measuring range for the measuring cycle. The state reached in step 104 is designated “D1” in FIG. 4.

All other measuring points approached in subsequent steps 108 to 130 are designated S1, S2, M1-M7, E1, E2, E3 in FIG. 4.

In the state set in step 106, the balloon probe 46 is clearly overstretched. This can be seen from the fact that in FIG. 4 the value of the esophageal pressure Peso detected in the balloon probe 46 is significantly higher than at all additional measuring points S1, S2, M1-M7, E1, E2, E3 approached.

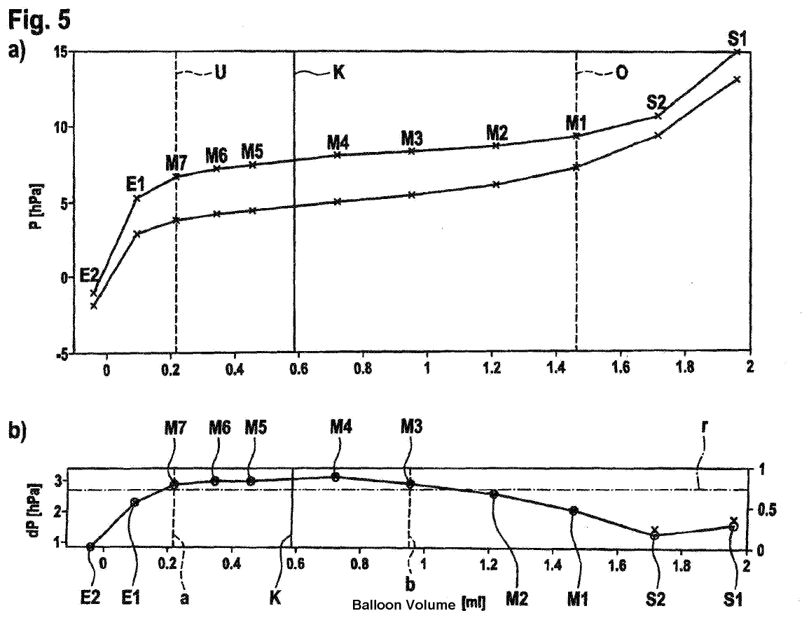

The upper limit of the measuring range is designated O in FIGS. 4 and 5, and the lower limit of the measuring range is designated U in FIGS. 4 and 5. It can be seen that in the calibration procedure shown in FIGS. 4 and 5, the lower limit U of the measuring range is at measuring point M7 and the upper limit O of the measuring range is at measuring point M1. The corresponding amount Vballoon of measuring fluid in the balloon probe 46 was selected as the reference quantity for the upper limit O and lower limit U in each case. O thus denotes the amount of measuring fluid in the balloon probe 46 at the measuring point M1, which corresponds to the upper limit of the measuring range. U denotes the amount of measuring fluid in the balloon probe 46 at the measuring point M7, which corresponds to the lower limit of the measuring range.

Starting from step 106, a predetermined amount of measuring fluid is pumped out from the balloon probe 46 in step 108. This sets an amount of measuring fluid in the balloon probe 46 at which a pressure Peso is detected that is slightly above the esophageal pressure expected in the measuring range (see measuring points M1 to M7). This state is designated “S1” in FIGS. 4 and 5. Alternatively, measuring fluid could be pumped out from the balloon probe 46 until a predetermined value of Peso is detected that is slightly above the esophageal pressure expected in the measuring range (see measuring points M1 to M7).

During the “S1” state, the esophageal pressure Peso is detected in the balloon probe 46 over a plurality of breathing cycles. It can be seen in FIG. 4 that the esophageal pressure Peso reflects the individual breathing cycles, but only to a very small extent, indicating low sensitivity of the balloon probe 46.

Starting from step 108, a predetermined amount of measuring fluid is pumped out from the balloon probe 46 in step 110, until the state designated “S2” in FIGS. 4 and 5 is reached. In step 110, the same procedure as described with respect to step 108 is repeated for the “S2” state. Again, only slightly pronounced breathing cycles can be seen and thus only low sensitivity of the balloon probe 46.

After completion of step 110, a predetermined amount of measuring fluid is again pumped out from the balloon probe 46 in step 112 until the state designated “M1” in FIGS. 4 and 5 is reached. The same procedure as described in relation to step 108 is repeated in step 112 for the “M1” state.

It can be seen in FIGS. 4 and 5 that in the state at measuring point M1 (and also in the subsequent states at measuring points M2 to M7) the esophageal pressure Peso clearly reflects the individual breathing cycles. Here, the maxima of the Peso curve are to be assigned to the esophageal pressures Peso_insp in the balloon probe 46 detected at the end of a respective inspiration phase, and the minima of the Peso curve are to be assigned to the esophageal pressures Peso_exp in the balloon probe 46 detected at the end of a respective expiration phase.

This procedure is subsequently repeated several times (see steps 114-130) to approach further measuring points M2-M7, E1, E2. At each of the further measuring points M2-M7, E1, E2, the esophageal pressure Peso in the balloon probe 46 is detected over a plurality of breathing cycles in the same manner as described with reference to steps 108, 110 and 112, and the maxima of the Peso curve are assigned to the esophageal pressures Peso_insp in the balloon probe 46 detected at the end of a respective inspiration phase and the minima of the Peso curve are assigned to the esophageal pressures Peso_exp in the balloon probe 46 detected at the end of a respective expiration phase.

The individual steps 110-130 are not shown in detail in the flowchart of FIG. 3.

On the basis of FIG. 4 it can be seen that these steps each belong to a specific amount of measuring fluid in the balloon probe 46, which are designated as measuring points S1, S2, M2-M7, E1, E2. The amount of measuring fluid in the balloon probe 46 is always changed in monotonic manner, in particular always decreased, during the transition from one of the measuring points S1, S2, M1-M7, E1, E2 to the next measuring point. In this case, the step width or increment, i.e. the amount by which the amount of measuring fluid in the balloon probe 46 is changed during the transition from one of the measuring points to the next measuring point, can always be the same. However, it is also conceivable that the amount by which the amount of measuring fluid in the balloon probe 46 is changed is chosen differently in each step, as long as the change always occurs in the same direction (i.e. the amount is always decreased or always increased). Since the assignment between a respective measuring point and the associated amount of measuring fluid in the balloon probe 46 is unique, for the sake of simplicity, in the following not only a respective measuring point is designated D1, S1, S2, M1-M7, E1, E2, etc, but rather the respective associated amount of measuring fluid in the balloon probe 46. M1, for example, designates both the measuring point M1 and the associated amount of measuring fluid in the balloon probe 46. This amount of measuring fluid in the balloon probe 46 also represents the upper limit O for the measuring range. In like manner, for example, M7 designates both the measuring point M7 and the associated amount of measuring fluid in the balloon probe 46. This amount of measuring fluid in the balloon probe 46 also represents the lower limit U for the measuring range. The same applies accordingly to the other measuring points M2-M6.

In FIG. 4, it can be seen that for the measuring points M1-M7, the detected esophageal pressure signal Peso clearly represents the individual breathing cycles, but that starting from the measuring point E1, the detected esophageal pressure signal Peso starts to become unstable. This indicates that we are now entering a range in which the balloon probe 46 is only insufficiently filled with measuring fluid and thus can no longer respond adequately to the pressure changes caused by the individual breathing cycles. At measuring points E2 and E3, it can be assumed that the balloon probe 46 has fully collapsed and the esophageal pressure Peso hardly indicates any breathing cycles. The measuring cycle is then terminated when measuring point E3 is reached. The measuring point E3 differs in so far as an esophageal pressure Peso is no longer detected and assigned at E3.

The state M1 designates the upper limit O of the measuring range that can be used for the measuring cycle. The actual calibration is therefore limited to this measuring range defined by the measuring points M1-M7. It can be seen in FIG. 4 that for all measuring points M1-M7 in the measurement range, the esophageal pressure Peso detected in the balloon probe is substantially in the same range. This indicates that the balloon probe 46 together with the esophageal wall now forms an essentially elastic system that expands or contracts according to the amount of measuring fluid in the balloon probe 46. These ratios remain the same until the lower limit U of the measuring range is reached when measuring point M7 is reached. In the measuring range between measuring points M1 and M7, and thus between values from the upper limit O to the lower limit U for the amount of measuring fluid in balloon probe 46, there is an approximately linear relationship between the respective predetermined amount of measuring fluid from the balloon probe 46 and the respective detected esophageal pressure Peso. The slope of a straight line representing this linear relationship is approximately the same for the relationship between the esophageal pressure Peso_ins detected at the end of the expiration phase and the predetermined amount of measuring fluid in the balloon probe 46, and for the relationship between the esophageal pressure Peso_exp detected at the end of the expiration phase and the predetermined amount of measuring fluid in the balloon probe 46. In the areas outside the measuring range (measuring points S1, S2 and measuring points E1, E2, respectively), the relationship between the respective predetermined amount of measuring fluid from the balloon probe 46 and the respective detected esophageal pressure Peso changes significantly. This relationship is no longer approximately linear in these areas and shows a much stronger increase or decrease in the detected esophageal pressure Peso with larger or smaller amounts of measuring fluid in the balloon probe 46. Also, the difference between the esophageal pressure Peso_insp detected for a predetermined amount of measuring fluid in the balloon probe 46 at the end of a respective expiration phase and the esophageal pressure Peso_exp detected at the end of a respective expiration phase becomes smaller very quickly in the areas (measuring points S1, S2 and E1, E2, respectively) lying outside the actual measuring range (measuring points M1 to M7).

FIG. 5 shows the significance of this calibration procedure. FIG. 5a shows schematically for each amount of measuring fluid Vballoon in the balloon probe 46, set as measuring points S1, S2, M1-M7, E1, E2 during a measuring cycle, the respective detected esophageal pressure Peso_insp in the balloon probe 46 at the end of the inspiration phase and the respective detected esophageal pressure Peso_exp in the balloon probe 46 at the end of the expiration phase. FIG. 5b shows the respective resulting differential pressure dP for each measuring point S1, S2, M1-M7, E1, E2 between the esophageal pressure in the balloon probe Peso_insp at the end of the inspiration phase and the esophageal pressure in the balloon probe Peso_exp at the end of the expiration phase.

It can be seen in FIG. 5 that within a range between the measuring points M1 to M7, the change in the detected esophageal pressure Peso_insp in the balloon probe 46 at the end of the inspiration phase and the detected esophageal pressure Peso_exp in the balloon probe 46 at the end of the expiration phase has an approximately linear relationship with the change in the amount of measuring fluid Vballoon in the balloon probe 46. The slope of the two resulting curves Peso/Vballoon is very flat and essentially the same for the esophageal pressure values at the end of the inspiration phase Peso_insp and the esophageal pressure values at the end of the expiration phase Peso_exp. Accordingly, it also results that the difference dP between the respective detected esophageal pressure Peso_insp in the balloon probe 46 at the end of the inspiration phase and the respective detected esophageal pressure Peso_exp in the balloon probe 46 at the end of the expiration phase remains substantially constant in the measuring range defined by the measuring points M1 to M7, while this difference dP quickly becomes very small in the ranges lying outside this measuring range, in which the measuring points S1, S2 and E1, E2 are located. It follows that an optimal calibration of the esophageal catheter 48 is at an amount of measuring fluid in the balloon probe 46 that lies within the measuring range defined by the measuring points M1 to M7, i.e. lies within the range of the amount of measuring fluid in the balloon probe 46, between the two vertical dotted lines O and U shown in FIG. 5a). Accordingly, as a result of the calibration, the calibration controller 60 selects an amount of measuring fluid in the balloon probe 46 that is within the measuring range of the measuring fluid defined by the measuring points M1 to M7. This amount is represented by the vertical line K in FIG. 5b) and is set as the amount of fluid in the balloon probe 46 after the calibration procedure is completed, as shown in the horizontally extending portion of the Vballoon curve designated K in the right portion of FIG. 4.

FIG. 5b) illustrates how the selection of the optimal amount of measuring fluid in the balloon probe 46 takes place within the measuring range defined by the measuring points M1 to M7. Within the measuring range, first the measuring point is searched for at which the difference dP between the respective detected esophageal pressure Peso_insp in the balloon probe 46 at the end of the inspiration phase and the respective detected esophageal pressure Peso_exp in the balloon probe 46 at the end of the expiration phase is maximum. In the example shown in FIG. 5b), this is the case at measuring point M4. Thereafter, a permissible fluctuation range around this maximum difference dPmax is defined. Values of the difference dP lying within this fluctuation range are considered as not significantly different from the maximum value dPmax. In the example shown in FIG. 5b), the permissible fluctuation range is 10% of the maximum difference dPmax determined at measuring point M4. This fluctuation range is indicated by the dash-dotted line r in FIG. 5b). The differences dP determined for the measuring points M3, M4, M5, M6 and M7 lie within the fluctuation range. All these measuring points are adjacent to the measuring point M4 and adjacent to each other. Therefore, a range of an amount of measuring fluid in the balloon probe 46, which lies between the measuring points M3 and M7, is considered equivalent with respect to a filling the balloon probe 46 with measuring fluid. This area is indicated in FIG. 5b) by vertical dashed lines a and b. The middle of the area between lines a and b is selected as the optimum amount of measuring fluid in the balloon probe 46. In this way, the optimum amount of measuring fluid in the balloon probe 46 is obtained as a result of calibration, see the vertical line K in FIG. 5b). This amount K is set as the amount of fluid in the balloon probe 46 after the calibration procedure is completed, see the rightmost part of the Vballoon curve in FIG. 4.

After completion of the calibration procedure, the calibration system 80 in step 132 again introduces an amount of measuring fluid into the balloon probe that is clearly above the amount corresponding to the measuring points M1 to M7 of the measuring range. In this step, overstretching of the balloon probe 46 is again to be brought about (in FIG. 4, this state is designated “D2”), before thereafter, in step 134, the calibrated state determined in the preceding calibration procedure, which is designated “K” in FIG. 4 (and in which, consequently, the balloon probe 46 is filled with the optimal amount K of measuring fluid), is set by withdrawing a corresponding amount of measuring fluid from the balloon probe 46.

Alternatively, it could also be envisaged that the measuring cycle shown in FIGS. 3 to 5 is followed by a further measuring cycle (or several further measuring cycles) before the calibrated state K is set. In such a case, the procedure returns from step 134 to step 108 and repeats the steps for the further measuring cycle analogously to steps 110-130 for the first measuring cycle. In FIG. 3, this is indicated by line 138.

Since the calibration controller 60 changes the amount of measuring fluid in the balloon probe 46 monotonically in at least two steps, starting from a start value S1 until an end value E3 is reached, in order to approach the respective measuring points S1, S2, M1-M7, E1, E2, the calibration can be completed in a short time, for example within only a few minutes. This allows the calibration to be repeated from time to time during an ongoing ventilation, thereby ensuring that the esophageal catheter 48 is always correctly calibrated, even if the optimal filling of the balloon probe 46 changes in the course of the ventilation. This permits ventilation of patients in automated ventilation modes for extended periods of time.

1. A calibration system for automatically setting an intended operational filling of an esophageal catheter with balloon probe, which can be inserted into the esophagus, for determining an esophageal pressure, in particular for a ventilation device, comprising:
a device for filling the balloon probe with a measuring fluid after placing the balloon probe in the esophagus,
a pressure sensor for detecting the esophageal pressure (Peso) prevailing in the balloon probe, and
a calibration controller which is designed such that it incrementally changes the amount of measuring fluid in the balloon probe, the calibration controller recording an esophageal pressure detected by the pressure sensor for each amount of measuring fluid in the balloon probe set incrementally as a measuring point in this way, and assigning the esophageal pressure to the respective set amount of measuring fluid in the balloon probe,
wherein the calibration controller is designed such that, for approaching the respective measuring points, it monotonically changes the amount of measuring fluid in at least two steps, starting from a start value until an end value is reached.

2. The calibration system according to claim 1,
further comprising a fluid draining device configured to drain measuring fluid from the balloon probe,
wherein the calibration controller controls the draining device for approaching the respective measuring points such that the amount of measuring fluid in the balloon probe decreases monotonically in at least two steps starting from the start value until the end value is reached.

3. The calibration system according to claim 2,
wherein the calibration controller is configured to control the arrangement for filling the balloon probe with a measuring fluid at least in a first measuring cycle for filling the balloon probe with an amount of measuring fluid that is greater than an upper limit of the measuring range between the start value and the end value.

4. The calibration system according to claim 1,
wherein the calibration controller is designed to execute at least two measurement cycles in succession.

5. The calibration system according to claim 4,
wherein the measuring range of the at least two successive measuring cycles is different, wherein a preceding measuring cycle determines the measuring range for a subsequent measuring cycle.

6. The calibration system according to claim 4,
wherein the calibration controller is designed such that it sets the distance between successive measuring points differently for the preceding measuring cycle and for the subsequent measuring cycle.

7. The calibration system according to claim 1,
wherein the calibration controller is configured to adaptively determine the increments between successive measuring points within the measuring range in a measuring cycle.

8. The calibration system according to claim 4,
wherein the calibration controller is designed such that the measuring fluid is not completely drained from the balloon probe between a preceding measuring cycle and a subsequent measuring cycle.

9. The calibration system according to claim 1,
wherein the calibration controller is designed such that for each measuring point, i.e. for each set amount of measuring fluid in the balloon probe, between the start value and the end value, it ascertains a respective measurement value for the esophageal pressure at the end of an inspiration phase and a measurement value for the esophageal pressure at the end of an expiration phase, and then determines the difference between the esophageal pressure at the end of the inspiration phase and the esophageal pressure at the end of the expiration phase.

10. The calibration system according to claim 9,
wherein the calibration controller is configured to determine a maximum value for the difference between the esophageal pressure at the end of the inspiration phase and the esophageal pressure at the end of the expiration phase within a range lying between the start value for the amount of measuring fluid in the balloon probe and the end value for the amount of measuring fluid in the balloon probe.

11. The calibration system according to claim 9, wherein the calibration controller is designed such that, for a respective measuring point between the start value and the end value, it ascertains the measurement value for the esophageal pressure at the end of an inspiration phase and the measurement value for the esophageal pressure at the end of an expiration phase during ongoing ventilation.

12. The calibration system according to claim 11, wherein the calibration controller is configured such that it compares the differences between the esophageal pressure at the end of the inspiration phase and the esophageal pressure at the end of the expiration phase, as determined for the respective measuring points, and then, when across a plurality of successive measuring points, the respective difference determined is within a predetermined fluctuation range about the maximum value, determines an optimum amount of measuring fluid in the balloon probe as an amount having a predetermined distance from an upper and/or lower edge of said plurality of successive measuring points.

13. The calibration system according to claim 1,
wherein the calibration controller is designed such that it ascertains a plurality of measurement values, in particular a plurality of pairs of measurement values, for the esophageal pressure at the end of an inspiration phase and for the esophageal pressure at the end of an expiration phase, for each measuring point between the start value and the end value, wherein the calibration controller determines an average and a statistical dispersion for the measurement value or pairs of measurement values for each measuring point on the basis of the plurality of measurement values or pairs of measurement values or a parameter derived therefrom, in particular the difference between the esophageal pressure at the end of the inspiration phase and the esophageal pressure at the end of the expiration phase, and determines the number of measurements per measuring point such that the average obtained can be regarded as statistically significant.

14. The calibration system according to claim 1,
wherein the calibration controller is configured to monitor for each measuring point between the start value and the end value whether the respective measurement of the esophageal pressure is affected by external circumstances, and to discard the respective measurement if such external circumstances are ascertained.

15. The calibration system according to claim 1,
wherein the calibration controller is designed to calculate a quality index based on the data ascertained during the calibration procedure.

16. The calibration system according to claim 1,
wherein the calibration controller is configured such that the esophageal pressure does not exceed a predetermined maximum pressure.

17. The calibration system according to claim 2,
wherein the calibration controller, when approaching the respective measuring points, controls the draining device such that the amount of measuring fluid in the balloon probe, starting from the start value, is incrementally reduced further and further until the end value is reached when a predetermined minimum end-expiratory esophageal pressure is reached or fallen short of.

18. A method for automated calibration of an intended operational filling of an esophageal catheter with balloon probe, which can be inserted into the esophagus, for determining an esophageal pressure, in particular for a ventilation device, comprising the following steps:
filling the balloon probe with a measuring fluid after placing the balloon probe in the esophagus,
detecting the esophageal pressure prevailing in the balloon probe, and
incrementally changing an amount of measuring fluid in the balloon probe, wherein for each amount of measuring fluid in the balloon probe set incrementally as a measuring point in this way, the esophageal pressure is detected and assigned to the respective set amount of measuring fluid in the balloon probe,
wherein, for approaching the respective measuring points, the amount of measuring fluid is changed monotonically in at least two steps starting from a start value until an end value is reached.

19. The method according to claim 18,
further comprising at least one additional method step implicitly mentioned with reference to a formation of a calibration system.

20. A computer program product containing program instructions, upon execution of which on a data processing system, in particular on a microprocessor or a microcontroller for controlling an esophageal catheter with balloon probe, a method according to claim 18 is carried out.
